(19) United States
(12) Patent Application Publication
(10) Pub. No.: US 2025/0377359 A1
(43) Pub. Date: Dec. 11, 2025

(54) Human Papillomavirus Multiplex Serology Assay

(71) Applicant: University of Maryland, Baltimore, Baltimore, MD (US)

(72) Inventors: Rebecca Gee Nowak, Washington, DC (US); Mohammad Sajadi, Cockeysville, MD (US); Maryam Karimi, Baltimore, MD (US); Arash Aslanabadi, Baltimore, MD (US)

(73) Assignee: University of Maryland, Baltimore, Baltimore, MD (US)

(21) Appl. No.: 19/230,235

(22) Filed: Jun. 6, 2025

Related U.S. Application Data
(60) Provisional application No. 63/656,822, filed on Jun. 6, 2024.

(51) Int. Cl. G01N 33/569; G01N 33/543; G01N 33/68

(52) U.S. Cl. CPC G01N 33/56983; G01N 33/54326; G01N 33/6854; G01N 2333/025; G01N 2469/20

(57) ABSTRACT

Provided herein are a platform and methods for multiplex serological assays to detect human papillomavirus antibodies in a subject and to measure antibody titer or antibody response. The platform and methods utilize recombinant viral antigenic proteins, for example, recombinant L1 proteins. Antibody titer or antibody response are categorized as one of a high, intermediate or low titer or response or no titer or response.

### CROSS-REFERENCE TO RELATED APPLICATIONS

This non-provisional patent application claims benefit of priority under 35 U.S.C. § 119 (e) of provisional application U.S. Ser. No. 63/656,822, filed Jun. 6, 2024, the entirety of which are hereby incorporated by reference.

### FEDERAL FUNDING LEGEND

This invention was made with government support under Grant Number DE031516 awarded by the National Institutes of Health. The government has certain rights in the invention.

### BACKGROUND OF THE INVENTION

### Field of the Invention

The present invention relates to the fields of virology and serology. More particularly, the present invention relates to serological assays and methods for determining the human papillomavirus antibody response in a subject.

### Description of the Related Art

Human papillomavirus (HPV) is one of the most common sexually transmitted infections worldwide with over 200 identified genotypes, of which 40 are transmitted through the genital tract and are associated with multiple types of malignancies (1,2). The virus can be transmitted through sexual contact (3), and persistent infection with high-risk HPV types leads to the development of precancerous lesions and, ultimately, invasive cancer (9).

High-risk HPV types, such as HPV-16 and HPV-18, are responsible for most cervical cancers, making HPV a major public health concern (4-6). Beyond cervical cancer, HPV is also involved in the development of other anogenital cancers, including cancers of the vulva, vagina, penis, and anus, as well as oropharyngeal cancers (8).

HPV vaccination programs have been highly successful in reducing the incidence of HPV infection and associated precancerous lesions, particularly among young women (10). Serologic testing for HPV antibodies is useful for monitoring population exposure to HPV, particularly in regions with low vaccine coverage or high HPV-related cancer rates (7). Since natural HPV infection is confined to the epithelium and infected cells are shed before cell death, it often triggers a minimal immune response, meaning not all infected individuals develop detectable antibodies (11). However, serological assays still provide valuable insight into past infections as well as vaccine-induced immunity, helping to inform public health strategies. Currently, such assays are primarily available in research settings.

Several serological methods have been developed to evaluate antibody responses to HPV, each with distinct benefits and limitations. One of the most widely used approaches is an enzyme-linked immunosorbent assay (ELISA) based on a virus-like particle (VLP) that strives to mimic the structure of the native virus (12). While these assays are highly specific, they are more difficult to produce, as they require the expression and assembly of multiple recombinant viral proteins in specialized cell lines. This makes the production process technically demanding and expensive, particularly when scaling up for large epidemiological studies. The process is also time-consuming, requiring careful quality control to ensure the correct assembly of virus-like particles. VLP-based assays are resource-intensive, requiring significant infrastructure and expertise (12). In addition, each HPV strain requires its own virus-like particle, limiting the flexibility of simultaneously detecting multiple strains.

GST (glutathione S-transferase)-fusion protein assays, used in multiplex serology, enable the simultaneous measurement of antibodies against multiple human papillomavirus types by fusing the L1 protein of human papillomavirus with a GST tag (13). However, this method introduces problems such as structural differences from native antigens, which can lead to non-specific binding and cross-reactivity (14), and decreased assay specificity. GST-tag may also interfere with antibody binding, limiting the assay sensitivity (15).

Competitive Luminex® (Luminex Corp.) immunoassays (cLIAs) are another method used for HPV serology, but come with significant challenges (16, 17). Designing and calibrating a cLIA is challenging due to the need for precise antigen optimization and competitor concentration. Small changes in these concentrations can lead to large variations in assay results, making calibration labor-intensive and time-consuming. Furthermore, competitive assays are prone to non-specific binding and cross-reactivity, subsequently complicating the interpretation of results and reducing accuracy.

Thus, the prior art is deficient in methods for assessing HPV-specific antibody responses and monitoring HPV immunity that overcomes current limitations. Particularly, the prior art is deficient in HPV multiplex serological assays that are cost-effective, scalable and highly accurate. The present invention fulfills this long-standing need and desire in the art.

### SUMMARY OF THE INVENTION

The present invention is directed to a platform for a multiplex serological assay to detect human papillomavirus antibodies. A plurality of spectrally unique microsphere bead sets are adhered magnetically to the platform. Recombinant viral antigenic proteins from at least one strain of human papillomavirus are covalently coupled to at least one of the spectrally unique microsphere bead sets.

The present invention also is directed to a method for detecting human papillomavirus antibodies in a subject. In the method a plasma sample is obtained from the subject and is added to the platform described herein. A fluorescently-labeled antibody directed against at least one human papillomavirus antibody is added. Fluorescent signals are monitored from each of the spectrally unique microsphere bead sets to which the fluorescently-labeled antibody is bound. The fluorescent signals detected from each of the spectrally unique microsphere bead sets are correlated to a specific human papillomavirus antibody present in the subject. The present invention is directed to a related method further comprising measuring a titer of each of the human papillomavirus antibodies present in the subject.

The present invention is directed further to a method for identifying at least one human papillomavirus genotype in a subject. In the method a plasma sample is obtained from the subject. Recombinant viral proteins specific to different human papillomavirus genotypes are covalently coupled to spectrally unique microsphere bead sets. The recombinant viral proteins are contacted with the plasma sample whereby human papillomavirus antibodies contained therein bind to their recombinant viral proteins and the human papillomavirus antibodies are contacted with fluorescently labeled antibodies directed against the HPV antibodies. Fluorescent signals from the spectrally unique microsphere bead sets are detected and the fluorescent signals from specific HPV antibodies are correlated to their human papillomavirus genotype, thereby identifying the human papillomavirus genotype.

The present invention is directed further still to a multiplex serological assay for evaluating an human papillomavirus antibody response to human papillomavirus genotypes associated with cervical cancer in a vaccinated subject. In the assay a plasma sample is obtained from the vaccinated subject. Recombinant L1 proteins derived from the human papillomavirus genotypes are covalently conjugated to a plurality of spectrally unique microsphere bead sets and the recombinant L1 proteins are contacted with the plasma sample to bind human papillomavirus antibodies therein to them. The human papillomavirus antibodies bound to the recombinant L1 proteins are contacted with a fluorescently-labeled antibody specific for the human papillomavirus antibodies and fluorescent signals emitted therefrom are detected. A net fluorescent intensity is calculated from the fluorescent signals for each type of human papillomavirus antibody and compared for each type of human papillomavirus antibody to a known fluorescent intensity for the human papillomavirus antibody to categorize the antibody response as a high response, an intermediate response, a low response, or no response.

Other and further aspects, features, benefits, and advantages of the present invention will be apparent from the following description of the presently preferred embodiments of the invention given for the purpose of disclosure.

### BRIEF DESCRIPTION OF THE DRAWINGS

The appended drawings have been included herein so that the above-recited features, advantages, and objects of the invention will become clear and can be understood in detail. These drawings form a part of the specification. It is to be noted, however, that the appended drawings illustrate preferred embodiments of the invention and should not be considered to limit the scope of the invention.

FIG. 1 shows a comparison of net median fluorescence intensity (Net-MFI) values between infants and children against HPV-11 L1. Each dot represents an individual sample, with horizontal lines indicating the median values. A significant difference in Net-MFI was observed between infants and children (P=0.0397). For the cut-off determination, both babies and children were included to capture the range of potential negative samples. After determining the MFIs of the negative panel, statistical analysis did not show any outliers >5 SD.

FIG. 2 shows the net-MFI distribution across antibody titer groups. The bar graph represents the median Net-MFI values for samples categorized into high, intermediate, low, and negative titer groups. Individual data points are shown for each sample within the respective groups. Error bars indicate the standard error of the mean (SEM). The high and intermediate titer groups exhibit significantly elevated (P-value <0.0001) Net-MFI values compared to the low titer and negative groups, reflecting varying levels of antibody responses.

FIGS. 3A-3L show the serological responses (FIGS. 3A-3B) and for IgG antibodies and IgA antibodies the cutoff values based on the mean and standard deviations (SD) are shown (FIGS. 3C and 3H), and sensitivity (FIGS. 3D-3E and 3I-3J) and specificity (FIGS. 3F-3G and 3K-3L) calculations are measured at various thresholds.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, the terms “consist of” and “consisting of” are used in the exclusive, closed sense, meaning that additional elements may not be included.

As used herein, the term “includes” or “including” refers to “including, but not limited to”. The terms “includes, “including” and “including, but not limited to” are used interchangeably.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

As used herein, the term “spectrally unique microsphere bead sets” or “spectrally unique microsphere beads” refer to carboxylated polystyrene microparticles, i.e., beads, color-coded into spectrally distinct sets or regions.

As used herein, the terms “subject” and “patient” are interchangeable and refer to any person who is tested for the presence of human papillomavirus or the antibodies thereof.

In one embodiment of the present invention, there is provided a platform for a multiplex serological assay to detect human papillomavirus antibodies, comprising a plurality of spectrally unique microsphere bead sets adhered magnetically to the platform; and recombinant viral antigenic proteins from at least one strain of human papillomavirus covalently coupled to at least one of the spectrally unique microsphere bead sets.

In this embodiment, the recombinant viral antigenic proteins may be recombinant L1 proteins. Also in this embodiment the human papillomavirus strain may be HPV-6, HPV-11, HPV-16, HPV-18, HPV-35, HPV-45, or HPV-58 or a combination thereof.

In another embodiment of the present invention, there is provided a method for method for detecting human papillomavirus antibodies in a subject, comprising obtaining a plasma sample from the subject; adding the plasma sample to the platform described supra; adding a fluorescently-labeled antibody directed against at least one human papillomavirus antibody; monitoring fluorescent signals from each of the spectrally unique microsphere bead sets to which the fluorescently-labeled antibody is bound; and correlating the fluorescent signals detected from each of the spectrally unique microsphere bead sets to a specific human papillomavirus antibody present in the subject.

In this embodiment, the human papillomavirus antibodies are IgG antibodies or IgA antibodies or a combination thereof. Further to this embodiment the method may comprise measuring a titer of each of the human papillomavirus antibodies present in the subject. In this further embodiment the measuring step may comprise calculating a net fluorescence intensity from the fluorescent signals emitted from the fluorescently labeled antibodies in each of the spectrally unique microsphere bead sets; and comparing the net fluorescence intensity calculated for the antibodies of a strain of human papillomavirus to a range of known fluorescence intensities for those human papillomavirus antibodies to identify the titer thereof.

In an aspect of this further embodiment the human papillomavirus antibody may be an HPV-6 IgG antibody, an HPV-11 antibody, an HPV-16 antibody, HPV-18 antibody, and HPV-45 antibody, or an HPV-58 antibody that comprises a high antibody titer group with net fluorescence intensities that range, respectively, from about 12623 to about 24425, from about 13633 to about 26327, from about 5363 to about 13059, from about 4437 to about 23901, from about 518 to about 2130, or from about 6075 to about 12370 or wherein each of the HPV antibodies is an IgA antibody and each IgA antibody has a net fluorescence intensity that ranges from about 3 to about 6312.

In another aspect of this further embodiment human papillomavirus antibody may be an HPV-6 IgG antibody, an HPV-11 antibody, an HPV-16 antibody, or an HPV-18 antibody that comprise an intermediate titer group with net fluorescence intensities that range from about 4887 to about 23262 for the HPV-6 IgG antibody and the HPV-11 IgG antibody, and, respectively, from about 3090 to about 8207, or from about 1998 to about 14557 for the HPV-11 antibody, the HPV-16 antibody, or the HPV-18 antibody or wherein each of these human papillomavirus antibodies is an IgA antibody with a fluorescence intensity that ranges from about 4 to about 1946.

In yet another aspect the HPV antibody may be an HPV-6 IgG antibody, an HPV-16 antibody or an HPV-18 antibody, that comprise a low antibody titer group, with net fluorescence intensities that range, respectively, from about 2784 to about 10278, from about 1765 to about 4014 or from about 1487 to about 4325. In yet another aspect a net fluorescence intensity less than 260 may indicate the subject is negative for HPV antibodies.

In both embodiments and aspects thereof, measuring the titer may be method for identifying at least one human papillomavirus genotype in a subject, comprising obtaining a plasma sample from the subject; covalently coupling recombinant viral proteins specific to different human papillomavirus genotypes to spectrally unique microsphere bead sets; contacting the recombinant viral proteins with the plasma sample whereby human papillomavirus antibodies contained therein bind to their recombinant viral proteins; contacting the human papillomavirus antibodies with fluorescently labeled antibodies directed against the HPV antibodies; detecting fluorescent signals from the spectrally unique microsphere bead sets; and correlating the fluorescent signals from specific human papillomavirus antibodies to their human papillomavirus genotype, thereby identifying the human papillomavirus genotype. effective for monitoring exposure of a population to human papillomavirus or durability of an antibody response in a vaccinated population over time.

In this embodiment the recombinant viral antigenic proteins may be recombinant L1 proteins. Also in this embodiment the human papillomavirus strain is HPV-6, HPV-11, HPV-16, HPV-18, HPV-35, HPV-45, or HPV-58 or a combination thereof.

In yet another embodiment of the present invention, there is provided a multiplex serological assay for evaluating an human papillomavirus (HPV) antibody response to human papillomavirus genotypes associated with cervical cancer in a vaccinated subject, comprising obtaining a plasma sample from the vaccinated subject; covalently conjugating recombinant L1 proteins derived from the HPV genotypes to a plurality of spectrally unique microsphere bead sets; contacting the recombinant L1 proteins with the plasma sample to bind HPV antibodies therein to the recombinant L1 proteins; contacting the HPV antibodies bound to the recombinant L1 proteins with a fluorescently-labeled antibody specific for said HPV antibodies; detecting fluorescent signals emitted therefrom; calculating a net fluorescent intensity from the fluorescent signals for each type of HPV antibody; and comparing the net fluorescent intensity for each type of HPV antibody to a known fluorescent intensity for said HPV antibody to categorize the antibody response as a high response, an intermediate response, a low response, or no response.

In this embodiment, the human papillomavirus associated with cervical cancer may be HPV-16 or HPV-18 or a combination thereof. Also in this embodiment the HPV antibody may be an IgG antibody or an IgA antibody or a combination thereof. In addition the HPV antibody may be an HPV-16 IgG antibody or an HPV-18 IgG antibody; wherein the high response is, respectively, a net fluorescence intensity of about 5363 to about 13059 or of about 4437 and 23901, the intermediate response is, respectively, a net fluorescence intensity of about 3090 to about 8207 or of about 1998 to about 14557, the low response is, respectively, a net fluorescence intensity of about 1765 to about 4014 or of about 1487 to about 4325, and the no response is a net fluorescence intensity less than 260. Furthermore, the human papillomavirus antibody may be an HPV-16 IgA antibody or an HPV-18 IgA antibody; wherein the high response for either is a net fluorescence intensity of about 3 to about 6312, the intermediate response for either is a net fluorescence intensity of about 4 to about 1946, and the no response is a net fluorescence intensity less than 260.

Provided herein is a serological assay and methods of using the same that utilizes recombinant viral proteins as antigens. For example, recombinant L1 proteins from individual human papillomavirus strains are covalently coupled to magnetic microsphere beads combined with Luminex multiplexing technology. This serological assay targets the detection of systemic HPV-specific IgGs and systemic HPV-specific IgA. The methods provided herein bypass the need for labor-intensive VLP production while maintaining the antigenic specificity of the L1 protein. The present methods combine high specificity with scalability, enabling efficient high-throughput analysis of serological responses across multiple human papillomavirus types for a variety of immunoglobulins.

Compared to other assays, the L1-based assay of the present invention may offer advantages in terms of specificity, sensitivity, and scalability. The L1-based assay demonstrated 100% sensitivity and specificity in detecting HPV-specific antibodies across multiple types, requiring minimal calibration. One notable finding is the cross-reactivity observed with HPV-35 in individuals vaccinated with the 9-valent HPV vaccine, which does not include HPV-35. HPV-35 cross-reactivity has been demonstrated in serodepletion and vaccine protection studies (21-23). Based on being part of the A9 group (HPV-16, HPV-31, HPV-33 and HPV-35) and serodepeletion studies, it is contemplated that antibodies against HPV-16, HPV-31, and HPV-33 lead to crossreactive antibodies against HPV-35 that were detected.

The following examples are given for the purpose of illustrating various embodiments of the invention and are not meant to limit the present invention in any fashion.

### Example 1

### Method & Materials

### Antigens and Microsphere Beads

Recombinant L1 proteins from seven human papillomavirus types (HPV-6, 11, 16, 18, 35, 45, and 58) were used as antigens for the serological assay. These proteins were covalently coupled with magnetic microspheres for use in the Luminex multiplex system. The coupling efficiency was optimized with 5 μg of antigen per bead set, corresponding to regions no. 12, 14, 19, 22, 27, 34, and 42 of internally labeled carboxylated microspheres.

The following proteins were used:

Antigens: Recombinant L1 proteins for HPV-6 and HPV-11 were purchased from Creative Diagnostics (Catalog #DAG-P2510 and #DAG-P2518, respectively). HPV-16 and HPV-18 L1 proteins were obtained from MyBioSource (Catalog #MBS318636 and #MBS318652, respectively). HPV-35, HPV-45, and HPV-58 L1 proteins were sourced from Creative Biomart (Catalog #HPV35-004H, #HPV45-005H, and #HPV58-002H).

Monoclonal Antibodies (mAbs): Monoclonal antibodies for HPV-6, HPV-11, HPV-16, and HPV-35 were obtained from Creative Diagnostics (Catalog #CABT-B880339, CABT-B8804, CABT-CS176, and CABT-CS276, respectively). The antibody for HPV-18 was purchased from Abbexa (Catalog #abx110595), the antibody for HPV-45 from Alpha Diagnostic (Catalog #HPV45L41-S), and the antibody for HPV-58 from Creative Diagnostics (Catalog #CABT-B8811).

### Positive Controls for IgG Assay:

Monoclonal antibodies (mAbs) against the L1 proteins of the respective HPV serotypes were used as positive controls, sourced as detailed above.

### Negative Controls for all Assays:

Negative controls consisted of plasma samples from infants aged between 1-2 years of age, and plasma samples from children aged 10-12, purchased from BocaBiolistics (Pompano Beach, FL). Older children were included as they are closer in age and immunologic status to adults, in order to better reflect the breadth of background antibody responses (and possible cross-reactivity) that could occur with exposure to different pathogens. Infants ages were chosen to preclude any remnant circulating maternal antibodies, and children's ages were chosen for low likelihood of vaccination, depending on geographic location and vaccination guidelines at the time of sampling. Both groups were low risk for HPV infection.

### Coupling of Target Antigens to Beads

The seven recombinant L1 proteins from human papillomavirus strains were conjugated to spectrally unique magnetic carboxylated microspheres using the Luminex coupling kit (Catalogue No: 40-50016). Each antigen was covalently coupled to a different bead set, corresponding to magnetic microspheres. The conjugation was performed based on the manufacturer's instructions, with some modifications detailed below.

Before the conjugation process, one to five million microspheres were prepared by sonicating (Bandelin, Germany, Model No: SONOREX DIGITEC DT 100 H) and vortexing (Fisher Scientific, USA, Cat No: 14-955-163). They were then placed on a magnetic rack, allowing the magnetic microspheres to adhere to the magnets. The beads were washed three times with activation buffer (0.1 M NaH₂PO₄, PH 6.2), resuspended, and prepared for activation.

For activation of the carboxyl groups on the bead surfaces and stabilization of the transient intermediate formation, 1-ethyl-3-(3-dimethyl aminopropyl) carbodiimide hydrochloride (EDC; ThermoScientific Pierce) and sulfo-N-hydroxysulfosuccinimide (sNHS; Thermo Scientific Pierce) were added to the bead suspension. The mixture was incubated on a tube mixer to promote efficient coupling.

Based on the manufacturer's recommendation, 5 μg of antigen per one million beads was used. Bead-protein conjugation was performed by incubating the microspheres with the corresponding proteins for 2 hours in the dark, at room temperature on a Multi-Purpose Tube Rotator (Fisherbrand™) set at 25 rpm. After the incubation, unbound antigens were removed by washing with Wash Buffer (Ingredients, Cat No. 11-25167), and active sites on the beads that were not coupled to antigens were blocked. The concentration of the microspheres was measured using a LUNA-FL™ cell counter, and the beads were stored at 4° C. in the dark until further use.

### Confirmation of Conjugation

To evaluate the efficiency of the antigen coupling, a confirmation assay was performed using serial dilutions of monoclonal antibodies or positive sample controls specific to each protein. These controls were also used to optimize the antigen coupling concentration for each bead region. Once optimized, both positive and negative samples were applied to set up screening conditions and assess the reactivity of the antigen-coated beads. The entire procedure followed the Luminex guidelines, as per the manufacturer's instructions.

### Multiplex Assay

The Multiplex HPV assay was run with a patient plasma sample or monoclonal antibody. Following bead preparation, 25 μL of the 10 μg/ml mAb or 1:100 diluted patient plasma was added to each well of a 96-well plate containing 50 μL of the bead suspension. While protecting from direct light, the plate was incubated for 1 hour at room temperature (RT) with gentle shaking (300-500 rpm), allowing the antibodies in the patient's serum to bind to the antigen-coupled beads. This shaking ensured proper mixing and optimal antibody binding. After the incubation, the plate was washed three times with 200 μL of PBS-TBN using a magnetic plate washer to remove unbound antibodies and plasma components. In each wash step, 200 L of PBS-TBN was added, followed by a 2-3 minute incubation before the buffer was aspirated. The magnetic plate washer holds the antigen-bound beads securely while washing away unbound material, maintaining the integrity of the assay. After washing, 50 μL of phycoerythrin (PE)-conjugated anti-human IgG (Millipore, SIG-HC19-PEIGG), was added to each well. The plate was then incubated for 30 minutes at room temperature with gentle shaking. Following the secondary antibody incubation, the plate was washed again three times and was read using the Luminex MagPix system, with the instrument set to measure 50 μL per well. The system ensures that at least 100 beads from each region are analyzed, yielding robust data. The PE-conjugated secondary antibody provides the fluorescence signal, which is detected and measured by the Luminex system. Data was acquired and analyzed using Luminex xPONENT software, which reports the results as median fluorescence intensity (MFI) values for each antigen-specific bead set.

Each assay plate included two wells containing only dilution buffer to serve as the blank signal. Additionally, corresponding monoclonal antibodies (mAbs) as positive controls and one constant negative control were included in every assay run to validate the performance of the test and ensure consistency across plates.

For test runs after the cut-off determination, minor variations between assay plates were controlled by adjusting MFIs based on the negative control signal from the cut-off run. Specifically, the percentage difference between the negative control from the cut-off run and the test run was applied to all the samples in the test run.

### Proficiency Panel Testing

To further validate the performance of the assay, it was tested on a blinded proficiency panel developed at NIH (18). This panel was designed to evaluate assay performance, specifically the ability to accurately measure negative, low, intermediate, and high levels of HPV type-specific IgG antibodies. The proficiency panel we used was made up of 28 HPV vaccinees and 12 negative controls.

### IgA Serology

For IgA serology, the same protocol outlined for IgG detection was followed, with minor adjustments. A phycoerythrin (PE)-conjugated anti-human IgA (Millipore, SIG-HC19-PEIGA) was used as the secondary antibody for detecting IgA responses. Due to the absence of established secondary monoclonal antibodies against human papillomavirus strains for IgA detection, we used plasma that had high IgA reactivity as our positive control. The negative control samples were identical to those used for IgG assays, consisting of plasma from individuals with no prior human papillomavirus exposure.

### Statistical Analysis and Cut-Off Determination

To establish the seropositivity threshold for each HPV protein type, cutoffs were determined by analyzing the Net-MFI (Median Fluorescence Intensity) values from the entire group of 26 infants and children. The mean and standard deviation (SD) of the Net-MFI values were calculated for each HPV type. To account for outliers, we planned to exclude any Net-MFI value exceeding the Mean+5 SDs for the IgA and IgG negative panels, with this process repeated iteratively until no further outliers were detected. Additionally, a negative control sample was run on both the assay plate and the proficiency plate. Net-MFI values for all samples were normalized based on the observed minor differences in the negative control sample across both plates, ensuring consistency and comparability of results.

Data analysis was performed using GraphPad Prism (v9.2.0, GraphPad Software, LLC). An unpaired t-test was used for two-group comparisons. Results are expressed as mean±SD, with statistical significance set at p<0.05.

### Example 2

### Results

### Cut-Off Determination

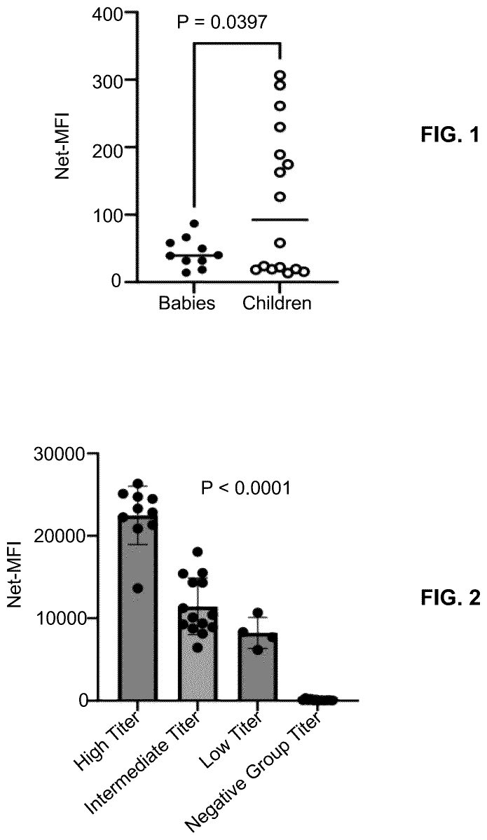

When determining the reactivity of plasma in the low-risk HPV group, there was a significant difference in the Net-MFI values between infants and children, with the children aged 10-12 having higher values than the infants for HPV-6, 11, 16, 35, and 58 (P<0.05) (FIG. 1). Including older children allowed for better calibration of the seropositivity threshold by reducing the likelihood of false-positive results in subsequent adult analyses.

### Sensitivity and Specificity

After setting the assay cut-off for each antigen above, the 40-sample proficiency panel was run on the HPV-6, 11, 16, 18, 35, 45, and 58 Luminex assay. The assay demonstrated high sensitivity, with 100% detection of antibody responses in individuals with known vaccination status for the included HPV types (HPV-6, 11, 16, 18, 45 and 58). Across all samples in the proficiency panel, the assay achieved 100% sensitivity for HPV-6, 11, 16, 18, 45, and 58 at the +3 standard deviations (SD) threshold. At +5 SD, sensitivity remained 100% for all HPV types mentioned HPV-45 dropping to 92.9%.

Because the NIH proficiency panel had not been validated for IgA, the sensitivity could not be calculated. However, the same algorithm for plasma IgG Net-MFI signals was applied to plasma IgA. Based on this, the percentage positive for HPV-6, 11, 16, 18, 45, and 58 were calculated as 64.3%, 96.4%, 75%, 67.9%, 57.1%, and 85.7%, respectively, at the Mean+3 SD threshold. At the Mean+5 SD threshold, the percent positive for plasma IgA detection for HPV-6, 11, 16, 18, 45, and 58 were calculated as 53.6%, 96.4%, 57.1%, 46.4%, 57.1%, and 60.7%, respectively. The percent positive for HPV-35 IgG and IgA was 50% and 14.3% at Mean+3 SD threshold and 35.7% and 14.3% at Mean+5 SD, respectively. Specificity was also high, with the assay correctly identifying all negative samples. At Mean+5 SD threshold specificity for HPV-6, 11, 16, 18, 35, 45 and 58 was calculated 100% and remained at 100% at Mean+3 SD for both plasma IgG and IgA, indicating the assay's robustness in distinguishing true negative samples from those with antibody responses to vaccination.

### High Antibody Titer Group

From the proficiency panel, 10 samples were classified as having high antibody titers. These individuals had all received the 9-valent HPV vaccine, which included L1 antigens from HPV-6, 11, 16, 18, 45, and 58. In these 10 samples, robust immune responses were detected across the included HPV types. In FIG. 2 Net fluorescence intensity (Net-MFI) values for HPV-6 ranged from 12623 to 24425, indicating strong seropositivity. For HPV-11, Net-MFI values varied from 13633 to 26327, while Net-MFI for HPV-16 ranged from 5363 to 13059. Similarly, HPV-18 showed Net-MFI values between 4437 and 23901, demonstrating the ability of the vaccine to generate strong antibody responses. The remaining types, HPV-45 and HPV-58, also exhibited high Net-MFI values ranging from 518 to 2130 and from 6075 to 12370, respectively. For the IgA assay, the net MFI values across all strains of HPV-6, 11, 16, 18, 45, and 58 ranged between 3 and 6312, reflecting the diversity in antibody response levels.

### Intermediate Antibody Titer Group

Fourteen samples were classified as having intermediate antibody titers. These individuals had detectable immune responses but at a reduced magnitude compared to the high-titer group, potentially due to waning immunity post-vaccination or another cause. MFI values in this group (FIG. 2) showed greater variability, with HPV-6 and HPV-11 yielding MFI values ranging from 4887 to 23262, and HPV-16 showing values from 3090 to 8207. For HPV-18, the MFI values ranged from 1998 to 14557. These results suggest that while antibody levels were lower, individuals in this group retained detectable immunity. Net-MFI for the IgA assay ranged from 4 to 1946 in the intermediate group (HPV-35 was excluded).

### Low Antibody Titer Group

Four samples were classified as having low antibody titers. In this group, MFI values (FIG. 2) for HPV-6 ranged from 2784 to 10278, while values for HPV-16 and HPV-18 were considerably lower, ranging from 1765 to 4014 for HPV-16 and 1487 to 4325 for HPV-18.

### HPV-Negative Group

The assay's specificity was validated using 12 samples that were confirmed to be negative for HPV-specific antibodies. These individuals had no known history of human papillomavirus exposure or vaccination. MFI values in this group (FIG. 2) were consistently low across all human papillomavirus types, with values ranging from 1 to 45 for HPV-6 and 4 to 260 for HPV-11. These low values confirmed the assay's ability to accurately differentiate between HPV-exposed and non-exposed individuals, with no false positives recorded among the HPV-negative controls.

### IgG and IgA Serological Responses

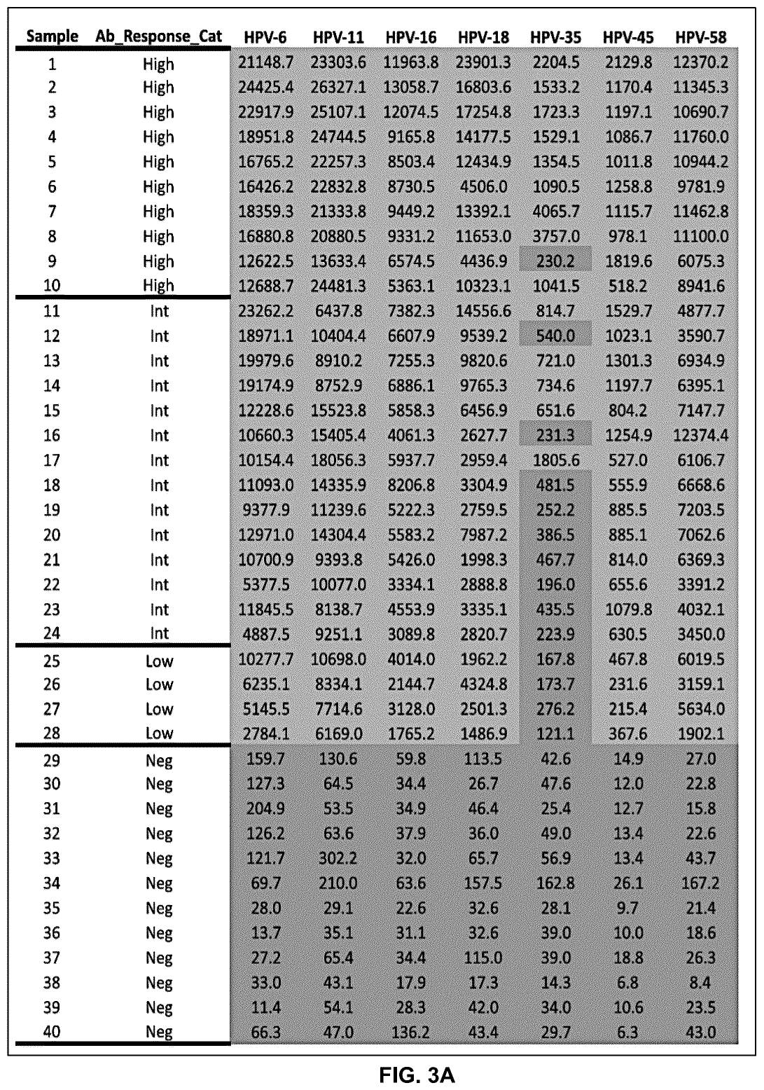

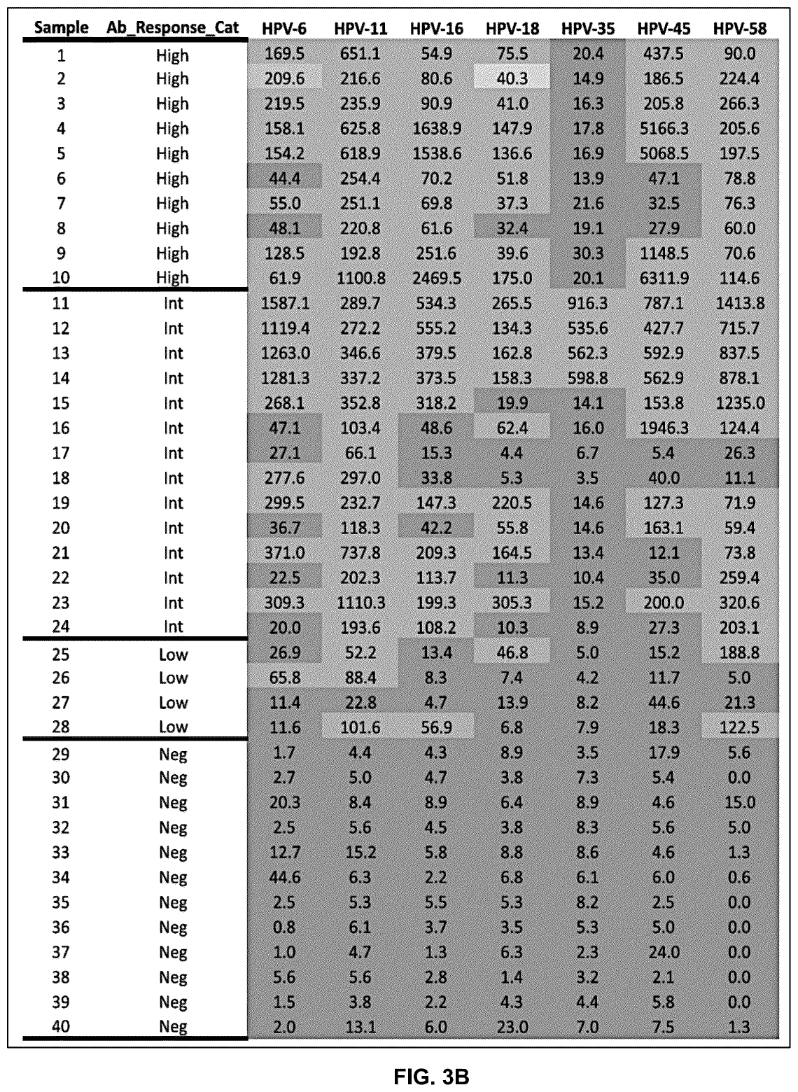

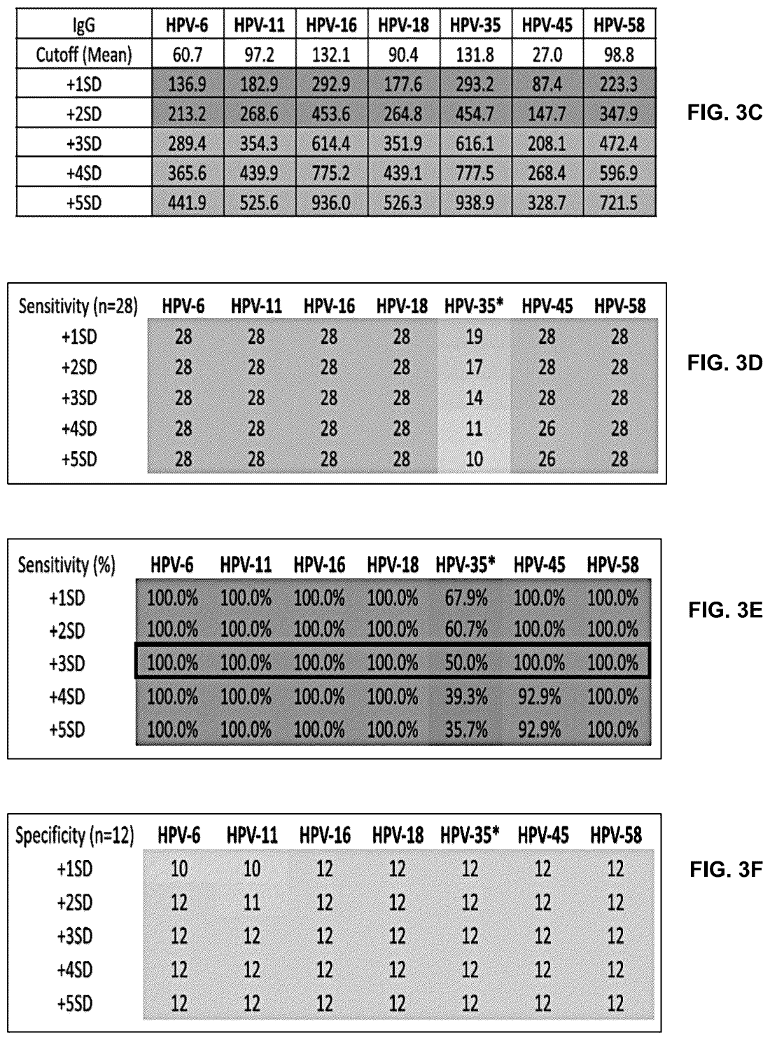

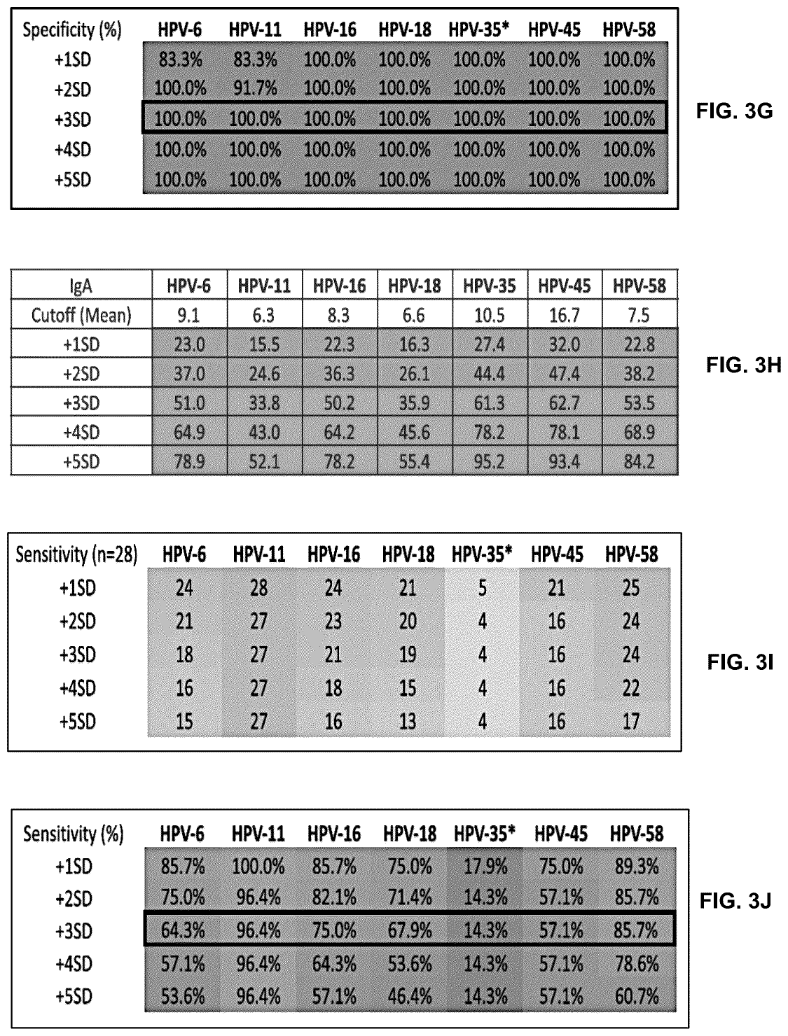

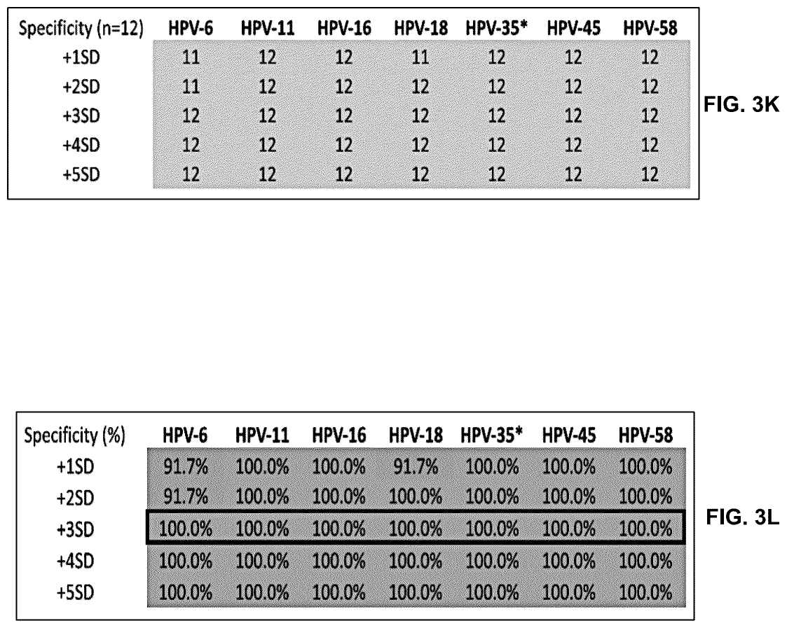

FIGS. 3A-3B are the serological responses measured for the immunoglobulins IgG and IgA in patient samples and categorized as high, intermediate (Int), low, or negative (Neg) responders. Heatmaps display individual MFI (median fluorescence intensity) values for each human papillomavirus type, with positive responses represented in light shading and negative responses in darker shading. FIGS. 3C-3G for IgG antibodies and FIGS. 3H-3L for IgA antibodies are the cutoff values based on the mean and standard deviations (SD), along with sensitivity and specificity calculations at various thresholds. Sensitivity and specificity percentages are presented for different SD cutoffs, highlighting assay performance in distinguishing seropositive and seronegative samples.

### REFERENCES

1. Centers for Disease Control and Prevention. Chapter 11: Human Papillomavirus. In: Meites E, Gee J, Unger E, Markowitz L, eds. Epidemiology and Prevention of Vaccine-Preventable Diseases. 14th ed. Atlanta, GA: CDC; 2021.
2. Oyouni A. A. A., Journal of infection and public health, 16 (4): 626-631 (2023).
3. Bruni et al., The Lancet Global Health, 11 (9): e1345-e1362 (2023).
4. Garcia et al., Sexually Transmitted Infections. In StatPearls. StatPearls Publishing, 2024.
5. Wang et al., Cancer letters, 471:88-102, (2020).
6. Okunade, K. S., Journal of Obstetrics and Gynaecology, 40 (5): 602-608 (2020).
7. Maver, P. J. and Poljak, M., Vaccine, 36 (36): 5416-5423 (2018).
8. Okunade K. S., Journal of Obstetrics and Gynaecology: The Journal of the Institute of Obstetrics and Gynaecology, 40 (5), 602-608 (2020).
9. Ye et al., MedComm, 4 (5): e368 (2023).
10. Rathod et al., Cureus, 15 (11): e49523 (2023).
11. Yokoji et al., Infectious Agents and Cancer, 18 (1): 70 (2023)
12. Faust et al., The Journal of general virology, 91 (Pt 7): 1840-1848 (2010).
13. Dias et al., Clinical and Vaccine Immunology, 12 (8): 959-969 (2005).
14. Anderson et al., PLOS Neglected Tropical Diseases, 9 (10): e0004168 (2015).
15. Robbins et al., BMC Infectious Diseases, 14:120 (2014).
16. Buck et al., Human Papillomaviruses: Methods and Protocols, pp. 445-462 (2006).
17. Zhang et al., Journal of Medical Virology, 95 (8): e29050 (2023).
18. Miller et al., Journal of Immunological Methods, 523:113585 (2023).
19. Pinto et al., Vaccine, 36 (32): 4792-4799 (2018).
1209. Muthamilselvan et al., Journal of Plant Biology, 66 (4): 331-340 (2023).
21. Boxus et al., Journal of virology, 90 (14): 6314-6325 (2016).
22. Combita et al., Journal of virology, 76 (13): 6480-6486 (2002).
23. Giroglou et al., Vaccine, 19 (13-14): 1783-1793 (2001).
24. Joshi et al., Vaccine, 41 (1): 236-245 (2023).
25. Clifford et al., Cancer Epidemiology Biomarkers & Prevention, 16 (9): 1874-1879 (2007).
26. Nowak et al., JAIDS Journal of Acquired Immune Deficiency Syndromes, 80 (4): 444-453 (2019).

What is claimed:

1. A platform for a multiplex serological assay to detect human papillomavirus antibodies, comprising:
a plurality of spectrally unique microsphere bead sets adhered magnetically to the platform; and
recombinant viral antigenic proteins from at least one strain of human papillomavirus covalently coupled to at least one of the spectrally unique microsphere bead sets.

2. The platform of claim 1, wherein the recombinant viral antigenic proteins are recombinant L1 proteins.

3. The platform of claim 1, where the human papillomavirus strain is HPV-6, HPV-11, HPV-16, HPV-18, HPV-35, HPV-45, or HPV-58 or a combination thereof.

4. A method for detecting human papillomavirus antibodies in a subject, comprising:
obtaining a plasma sample from the subject;
adding the plasma sample to the platform of claim 1;
adding a fluorescently-labeled antibody directed against at least one human papillomavirus antibody;
monitoring fluorescent signals from each of the spectrally unique microsphere bead sets to which the fluorescently-labeled antibody is bound; and
correlating the fluorescent signals detected from each of the spectrally unique microsphere bead sets to a specific human papillomavirus antibody present in the subject.

5. The method of claim 4, wherein the human papillomavirus antibodies are IgG antibodies or IgA antibodies or a combination thereof.

6. The method of claim 4, further comprising measuring a titer of each of the human papillomavirus antibodies present in the subject.

7. The method of claim 6, wherein the measuring step comprises:
calculating a net fluorescence intensity from the fluorescent signals emitted from the fluorescently labeled antibodies in each of the spectrally unique microsphere bead sets; and
comparing the net fluorescence intensity calculated for the antibodies of a strain of human papillomavirus to a range of known fluorescence intensities for those human papillomavirus antibodies to identify the titer thereof.

8. The method of claim 7, wherein the human papillomavirus antibody is an HPV-6 IgG antibody, an HPV-11 antibody, an HPV-16 antibody, HPV-18 antibody, and HPV-45 antibody, or an HPV-58 antibody that comprise a high antibody titer group with net fluorescence intensities that range, respectively, from about 12623 to about 24425, from about 13633 to about 26327, from about 5363 to about 13059, from about 4437 to about 23901, from about 518 to about 2130, or from about 6075 to about 12370 or wherein each of the human papillomavirus antibodies is an IgA antibody and each IgA antibody has a net fluorescence intensity that ranges from about 3 to about 6312.

9. The method of claim 7, wherein the human papillomavirus antibody is an HPV-6 IgG antibody, an HPV-11 antibody, an HPV-16 antibody, or an HPV-18 antibody that comprise an intermediate titer group with net fluorescence intensities that range from about 4887 to about 23262 for the HPV-6 IgG antibody and the HPV-11 IgG antibody, and, respectively, from about 3090 to about 8207, or from about 1998 to about 14557 for the HPV-11 antibody, the HPV-16 antibody, or the HPV-18 antibody or wherein each of these human papillomavirus antibodies is an IgA antibody with a fluorescence intensity that ranges from about 4 to about 1946.

10. The method of claim 7, wherein the human papillomavirus antibody is an HPV-6 IgG antibody, an HPV-16 antibody or an HPV-18 antibody, that comprise a low antibody titer group, with net fluorescence intensities that range, respectively, from about 2784 to about 10278, from about 1765 to about 4014 or from about 1487 to about 4325.

11. The method of claim 7, wherein a net fluorescence intensity less than 260 indicates the subject is negative for human papillomavirus antibodies.

12. The method of claim 6, wherein measuring the titer is effective for monitoring exposure of a population to human papillomavirus or durability of an antibody response in a vaccinated population over time.

13. A method for identifying at least one human papillomavirus genotype in a subject, comprising:
obtaining a plasma sample from the subject;
covalently coupling recombinant viral proteins specific to different human papillomavirus genotypes to spectrally unique microsphere bead sets;
contacting the recombinant viral proteins with the plasma sample whereby human papillomavirus antibodies contained therein bind to their recombinant viral proteins;
contacting said human papillomavirus antibodies with fluorescently labeled antibodies directed against the human papillomavirus antibodies;
detecting fluorescent signals from the spectrally unique microsphere bead sets; and
correlating the fluorescent signals from specific human papillomavirus antibodies to their human papillomavirus genotype, thereby identifying the human papillomavirus genotype.

14. The method of claim 13, wherein the recombinant viral antigenic proteins are recombinant L1 proteins.

15. The method of claim 13, where the human papillomavirus strain is HPV-6, HPV-11, HPV-16, HPV-18, HPV-35, HPV-45, or HPV-58 or a combination thereof.

16. An assay for evaluating an human papillomavirus antibody response to human papillomavirus genotypes associated with cervical cancer in a vaccinated subject, comprising:
obtaining a plasma sample from the vaccinated subject;
covalently conjugating recombinant L1 proteins derived from the human papillomavirus genotypes to a plurality of spectrally unique microsphere bead sets;
contacting the recombinant L1 proteins with the plasma sample to bind HPV antibodies therein to the recombinant L1 proteins;
contacting the human papillomavirus antibodies bound to the recombinant L1 proteins with a fluorescently-labeled antibody specific for said HPV antibodies;
detecting fluorescent signals emitted therefrom;
calculating a net fluorescent intensity from the fluorescent signals for each type of human papillomavirus antibody; and
comparing the net fluorescent intensity for each type of human papillomavirus antibody to a known fluorescent intensity for said human papillomavirus antibody to categorize the antibody response as a high response, an intermediate response, a low response, or no response.

17. The assay of claim 16, wherein the human papillomavirus associated with cervical cancer is HPV-16 or HPV-18 or a combination thereof.

18. The assay of claim 16, wherein the human papillomavirus antibody is an IgG antibody or an IgA antibody or a combination thereof.

19. The assay of claim 18, wherein the human papillomavirus antibody is an HPV-16 IgG antibody or an HPV-18 IgG antibody; wherein said high response is, respectively, a net fluorescence intensity of about 5363 to about 13059 or of about 4437 and 23901, said intermediate response is, respectively, a net fluorescence intensity of about 3090 to about 8207 or of about 1998 to about 14557, said low response is, respectively, a net fluorescence intensity of about 1765 to about 4014 or of about 1487 to about 4325, and said no response is a net fluorescence intensity less than 260.

20. The assay of claim 18, wherein the human papillomavirus antibody is an HPV-16 IgA antibody or an HPV-18 IgA antibody; wherein said high response for either is a net fluorescence intensity of about 3 to about 6312 and said intermediate response for either is a net fluorescence intensity of about 4 to about 1946, and said no response is a net fluorescence intensity less than 260.
